(19) United States
(12) Patent Application Publication
(10) Pub. No.: US 2006/0094083 A1
(43) Pub. Date: May 4, 2006

(54) Probiotic microorganisms producing chimeric human growth hormone fused with Fc fragment of human IgG for oral delivery system and methods for producing them

(76) Inventors: Yun-Jaie Choi, Seoul (KR); Chang-Hoon Lee, Iksan-si (KR); Jung-Hee Woo, Seoul (KR); Seung-Ha Kang, Suwon-si (KR); Seung-Hoon Choi, Seoul (KR); Sang-Kee Kang, Seoul (KR)

Correspondence Address: NATH & ASSOCIATES, PLLC, Sixth Floor, 1030 15th Street, N.W., Washington, DC, 20005, US

(21) Appl. No.: 11/211,464

(22) Filed: Aug. 26, 2005

(30) Foreign Application Priority Data
Nov. 3, 2004 (KR) 10-2004-0089046

(51) Int. Cl. C12P 21/06; C12N 1/21; C12N 1/18; C07K 14/61

(52) U.S. Cl. 435/69.4; 435/252.3; 435/254.23; 530/399

(57) ABSTRACT

The present invention relates to probiotic microorganisms producing chimeric human growth hormone for oral use and methods for preparing them.

The invention provides probiotic Lactobacillus or yeast transformant expressing chimeric protein which is human growth hormone fused with Fc fragment of human IgG, in which the transformants are safely delivered into intestine though oral route. Also, the invention provides a chimeric protein-expressing vector which can induce transcytosis in intestine epithelial cells.

Accordingly, the invention demonstrates that the chimeric protein for oral delivery system can be absorbed in intestine, and delivery of the chimeric protein by oral route using Lactobacillus has very excellent efficiency in vivo test in rats. Accordingly, the Lactobacillus of the present invention is an excellent deliverer of protein drugs.

### TECHNICAL FIELD

The present invention relates to probiotic microorganisms producing chimeric human growth hormone fused with Fc fragment of human IgG for oral delivery system and methods for producing them. More specifically, the invention relates to the probiotic microorganisms which are Lactobacillus or yeast transformant producing chimeric proteins (or fusion proteins), human growth hormone fused with Fc fragment of human IgG, in which the microorganisms are safely delivered into intestine by oral route, and then can be absorbed in the body.

### BACKGROUND ART

Recently, protein drugs such as growth hormone have been developed rapidly due to less toxicity than existing chemical drugs and bio-friendly properties. However, because protein drugs have low permeability into bio-membrane due to degrading easily by bio-enzymes and its large size, new drug delivery system to solve the problem of protein drugs needs developing. Drugs administration methods for treatment of disease or wounding or promotion of health comprise injection, oral, ointment, patch, etc. Injection of these methods has been widely used because of immediate remedial result of drug. However, injection induces pain and hyperactivity in patients by frequent injection. As a result, patients have been under a lot of stress and the method is too convenience. On the other hand, oral administration is very convenient, acceptable and economical because of patient's low detestation and no needs of a skilled person or special apparatus containing needle or Ringer's solution. However, if protein drugs are administrated orally, the drugs might loss most of their activity by attack of gastric acid or many kinds of digestive enzymes secreted from intestine going through digestive organ in the body. Though part of the drugs with whole activity is delivered into intestine, it is difficult to function properly due to being adsorbed in the body by effective intercept of intestinal epithelial cell layer which receives selectively foreign material. Accordingly, inconvenience injection is used so far for administration of drugs. As stated in the above, oral administration is the best ideal way for patient's self-administration because of convenience of administration and patient's adaptability to take drugs. Nevertheless, adsorption rate of protein drugs by oral route is one percentage and below. And, problems for oral use in instability as going through digestive duct and low permeability into intestinal mucous membrane have not been solved yet. According to biotechnology has been made rapid progress in recent years, various protein drugs are developing as future generation of drugs to replace chemical drugs. Therefore, it is urgent for development of new oral delivery system which can deliver stably in the body to increase substantially use of various protein drugs developed.

While, studies for lactic bacterium have been accelerated by the research results accumulated so far and studies for high value-added goods using lactic bacterium have been made process actively. For last twenties years, studies for lactic bacterium in genetic engineering have been mostly focused on Lactococcus (Lactococcus lactis ssp. lactis, Lactococcus lactis ssp. cremoris). In recent years, study for Lactobacilli has been made actively process, and characterization of various plasmids isolated from them and development of vectors are made process. Lactobacilli are used in inactivation of specific gene by genes engineering or insertion of genes by plasmids or insertion of genes into chromosomal DNA. Also, use of Lactobacilli goes on increasing widely from industrial field to medical field in recent years, because of their non-pathogenic characteristic. Development of recombinant Lactobacillus, due to their wide use range, is in proceeding by many researchers. In recent years, interest in live mucosal vaccine vector or oral delivery system is increasing. However, for the oral delivery system, effective and stable expression system to produce recombinant foreign protein in Lactobacillus needs. Though there were expression results of some kind of foreign proteins, their expressed amount was not enough to use industrially.

Accordingly, to solve these problems, the present inventors developed new oral delivery system with high-efficiency which can go through digestive duct safely, in which the delivery system was prepared as functional probiotic microorganisms which were transferred a peptide ligand as gene form in Lactobacillus by inserting the peptide ligand permeating intestinal mucous membrane by receptor mediated endocytosis into protein drugs.

### DISCLOSURE OF THE INVENTION

An object of the present invention is to provide probiotic microorganisms for oral use which deliver safely macromolecules such as growth hormone into intestine by oral route, and the molecules can be adsorbed in the body, and methods for preparing the microorganisms.

The object of the present invention is achieved by developing probiotic microorganisms producing chimeric human growth hormone for oral use, in which the microorganisms are prepared by constructing chimeric proteins which are human growth hormone fused with CH2 and CH3 domain of human IgG inducing transcytosis in intestinal epithelial cell; by preparing Lactobacillus and Pichia pastoris transformant producing the chimeric protein using Pichia pastoris expression system and Lactobacillus expression vector system; and, by testing their transcytotic activity, productivity of IGF1 and in vivo experiment in rats.

In one respect of the present invention, the invention provides a recombinant Lactobacillus expression vector which named pNZ123(1-5N) and illustrated by the map of FIG. 2, comprising human growth hormone coding gene conjugated with hinge-CH2-CH3 domain of Fc fragment of human IgG coding gene, in which the vector is derived from plasmid pNZ123 obtained from Lactobacillus brevis ATCC 8287.

In other respect of the present invention, the invention provides a Lactobacillus brevis H6(accession number: KACC 91137) expressing a chimeric protein which is human growth hormone fused with hinge-CH2-CH3 domain of Fc fragment of human IgG.

In other respect of the present invention, the invention provides a Pichia pastoris H6(accession number: KACC 93017) expressing a chimeric protein which is human growth hormone fused with CH2-CH3 domain of Fc fragment of human IgG.

In other respect of the present invention, the invention provides a Pichia pastoris H6(accession number: KACC 93018) expressing the chimeric protein which is human growth hormone fused with hinge-CH2-CH3 domain of Fc fragment of human IgG.

In other respect of the present invention, the invention provides preparation methods of probiotic microorganisms producing chimeric human growth hormone for oral use, in which the microorganisms are obtained by constructing recombinant expression vector, pNZ123(1-5N) containing human growth hormone and transcytotis inducing domains, and by transforming the vector into Lactobacillus.

A greater understanding of the present invention and its concomitant advantages will be obtained by referring to the following Experimental example provided, but it is not limit the scope of the present invention.

### BRIEF DESCRIPTION OF THE DRAWINGS

Other objects and aspects of the present invention will become apparent from the following description of embodiments with reference to the accompanying drawing in which:

FIG. 1 shows gene construction of fusion proteins which are human growth hormone conjugated with Fc fragment of human IgG.

FIG. 2 shows a map and construction of pNZ123(1-5N) Lactobacillus expression vector.

FIG. 3 is a gel photograph which shows Lactobacillus transformant identified by colony PCR.

FIG. 4 shows results of SDS-PAGE(a) and Western-blotting (b) of H6 fusion protein (human growth hormone-hinge-Fc fragment of IgG) secreted by Lactobacillus brevis.

FIG. 5 shows a procedure inserting the fusion protein-coding gene into yeast expression vector, pPICZ.

FIG. 6 shows results of SDS-PAGE(a) and Western-blotting (b) of H6 fusion protein(human growth hormone-hinge-Fc fragment of IgG) secreted by yeast.

FIG. 7 shows construction of transwell system to assay transcytotic activity of the probiotic microorganisms according to the present invention and a method of transcytosis assay using the same.

FIG. 8 represents results of transcytosis assay by ELISA (a) and Western-blotting (b) of H6 fusion protein secreted by yeast.

FIG. 9 shows a result of IGF1 production response in HepG2 cell cultured in a medium, supplemented with the fusion protein expressed from yeast strain.

FIG. 10 represents measurement results of transcytotic activity and IGF1 production response of the fusion protein produced from Lb. H6(Lactobacillus brevis H6 transformant).

FIG. 11 represents results of in vivo experiment which shows effect of Lb. H6 on the growth of rats.

### BEST MODE FOR CARRYING OUT THE INVENTION

### EXAMPLE I

### Construction and Expression of Candidate Protein Population Inducible Transcytosis in Intestinal Epithelial Cell

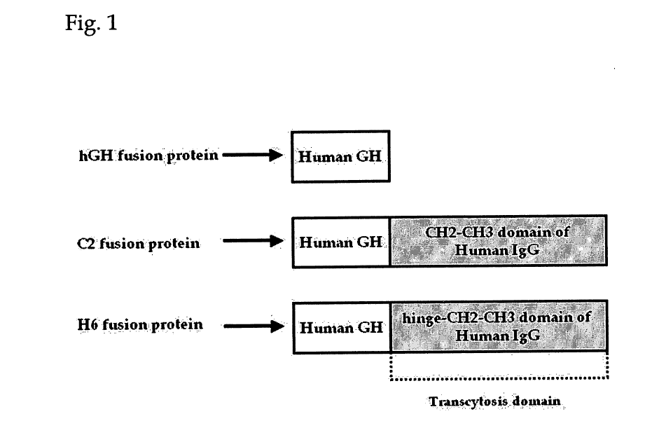

A candidate protein population inducible transcytosis in intestinal epithelial cell was constructed to prepare probiotic microorganisms for oral use. As shown in FIG. 1, Lactobacillus expression vector, pLb-H6 was constructed, which ligated the gene construct for mature human growth hormone conjugated with Fc fragment of human IgG1 as transcytosis domain. C2 fusion protein is the mature human growth hormone gene sequence conjugated with transcytosis domain, Fc fragment of human IgG without hinge region, CH2-CH3 domain. H6 fusion protein is the mature human growth hormone gene sequence conjugated with transcytosis Fc fragment of human IgG with hinge region sequence. The Lactobacillus brevis strain ATCC8287 was used as a host for expression plasmid vector and DNA for mature human growth hormone was amplified by PCR with Vent DNA polymerase.

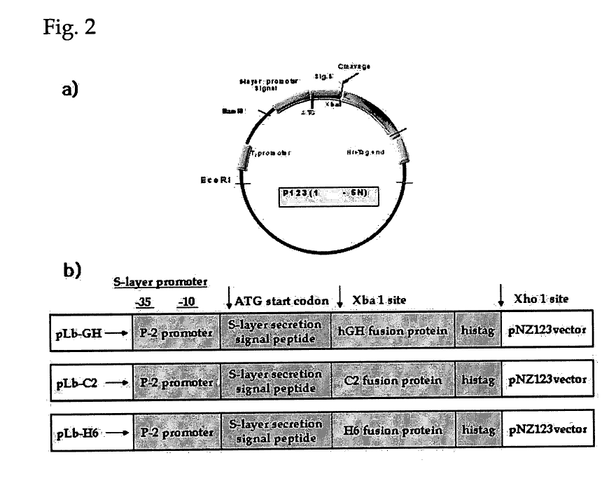

FIG. 2 shows Lactobacillus expression vector, pNZ123 derived vector, pNZ123(1-5N) which was the Lactobacillus secretion signal fused into the Lactobacillus expression vector(pLb-H6). This vector comprised P-2 promoter region of S-layer, secretion signal sequence and cloning sites (Xba I and Xho I). And, three types of fusion proteins are inserted in the pNZ123(1-5N) Lactobacillus expression vector.

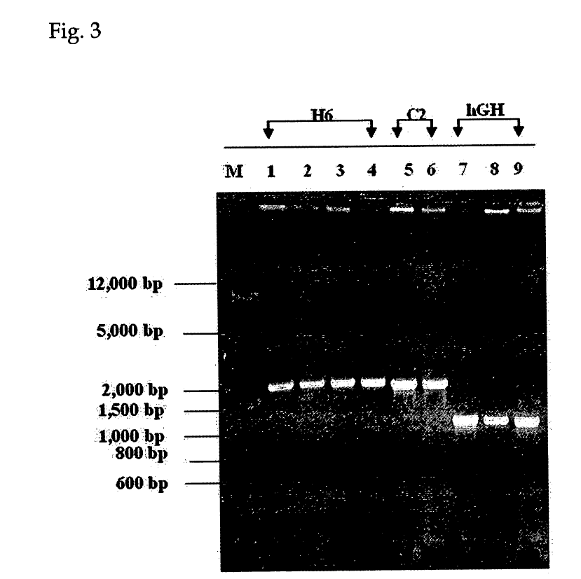

The L. brevis strain ATCC 8287 was transformed with the plasmid vector pLb-H6, pLb-C2 and pLb-GH in FIG. 2 by electroporation. The specific settings for electroporation were 2.5 kV, 400 Ohm and 25 uF. Immediately after transformation, the cells were allowed to recover for 2 h at 37° C. in MRS medium (Difco, USA), before plating onto selective(5 μg/ml chloramphenicol) MRS agar plates. Transformants were detected by colony PCR method in FIG. 3. Lane 1 to 4 represent Lactobacillus brevis transformed with pLb-H6 plasmid vector, Lane 5 and 6 represent Lactobacillus brevis transformed with pLb-C2 plasmid vector, and Lane 7 to 9 represent Lactobacillus brevis transformed with pLb-GH plasmid vector.

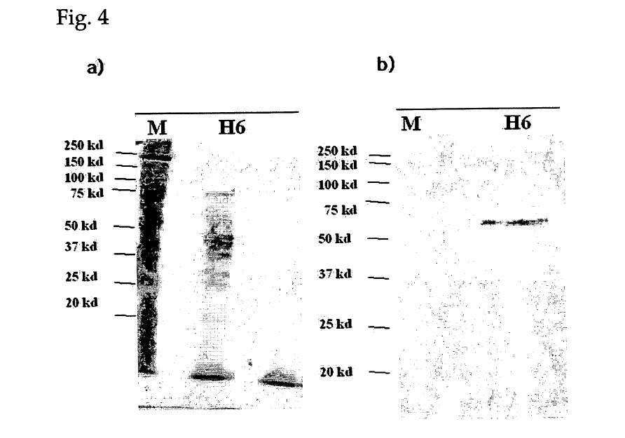

For the analysis of expression and secretion of the chimeric protein, the selected transformants were routinely grown in MRS medium supplemented with 5 μg/ml chloramphenicol at 37° C. under shaking. The cells were collected and dissolved in buffered MRS supplemented with 5 μg/ml chloramphenicol. Cells and culture supernatants were separated by centrifugation at 1,500 xg for 10 min. The proteins were extracted from the culture supernatant with ammonium sulfate precipitation method. The cell pellets were dissolved in TGE buffer and treated with mutanolysin and lysozyme as described previously. Protein fractions were separated by Sodium Dodecyl Sulphate Poly Acrylamide Gel Electrophoresis(SDS-PAGE) and electroblotted on a PVDF membrane. hGH conjugated with Fc fragment, chimeric protein was detected by immunoblotting with monoclonal mouse anti hGH as a primary antibody at a 1/5000 dilution. The secondary antibody was goat anti mouse IgG conjugated with HRP. FIG. 4 shows SDS-PAGE (a) and Western-blotting (b) of H6 fusion protein (human growth hormone-hinge-Fc fragment of IgG) secreted by Lactobacillus brevis. The expression of the protein by the above methods was observed.

### EXPERIMENTAL EXAMPLE I

### Test of Transcytosis Activity of the Chimeric Proteins Expressed in Yeast Expression System

To test the transcytosis capacity of the proteins carried out the transcytosis assay using transwell system. First, the proteins using P. pastoris over expression system were prepared, because it is difficult to gain abundant in protein in L. brevis expression system.

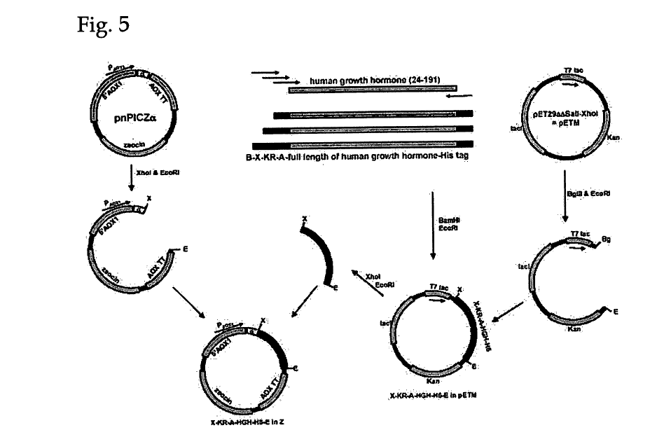

For this, coding genes of candidate proteins were constructed and inserted into PicZa vector (see FIG. 5). The proteins were prepared by methanol induction and detected using SDS-PAGE and western blotting.

The chimeric proteins to test their function were concentrated with ammonium sulfate precipitation method and dialyzed in HBSS at pH6.0, and purified with affinity chromatography using NTA column(Merck) and Protein G column (Millipore).

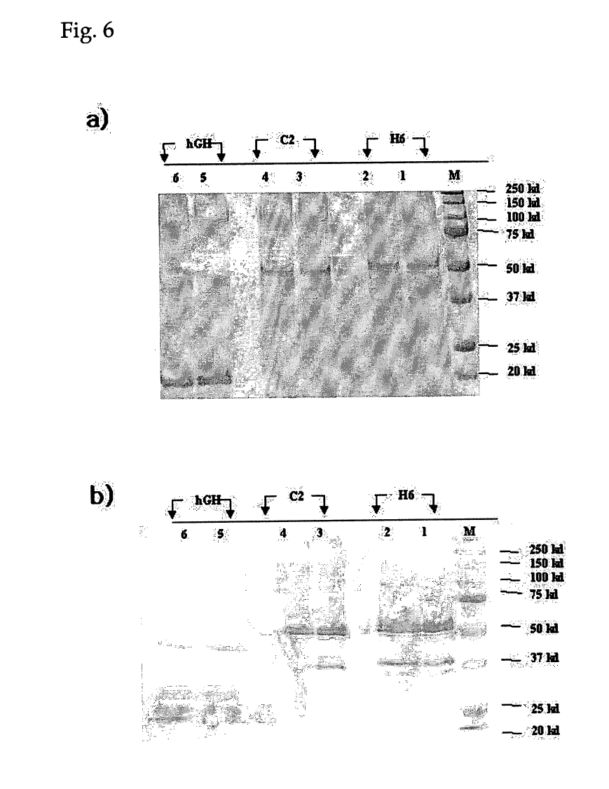

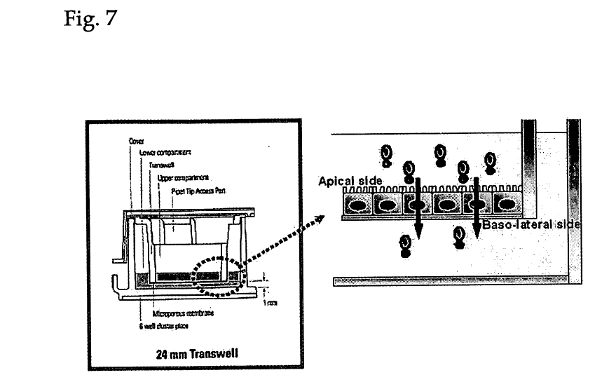

FIG. 6 shows chimeric proteins expressed in Pichia pastoris. Lane 1 and 2 represent H6 fusion protein(hGH-hinge-Fc-histag) is about 55 kDa size. Lane 3 and 4 represent C2 fusion protein (hGH-Fc without hinge region-histag) was about 52 kDa size. Lane 5 and 6 represent hGH chimeric protein(hGH-histag) was about 22 kDa size detected on SDS-PAGE (a) and western blotting (b) Also, to confirm the capability of transcytosis of the chimeric proteins in human intestinal cell line, confluent T84 monolayers to induce polarization were incubated on 3.0 μm pore sized transwell (from corning costar) shown in FIG. 7 until they were exhibited high electrical resistances(800-1,200Ω/cm²) in DMEM/F12 supplemented with 5% fetal calf serum (all from Gibco). Polarization of cell is the procedure that distribution of receptor or enzyme or other proteins leans to apical side or basolateral side. As a result, the cell resembles in vivo cell in function. To confirm polarization measured electrical resistance by OVM electrometer. T84 monolayers exhibiting high electrical resistances(800-1,200Ω/cm²) detected under OVM electrometer were equilibrated in HBSS and 1 μg fusion proteins (H6, C2, hGH) were added to the apical reservoirs respectively. 0.5% gelatin (sigma Chemical Co.) as a nonspecific blocker was added in ligand. Monolayers were incubated for 1 hour with the ligand at either 37° C. or 4° C., after which an aliquot of the contralateral well buffer was collected and were analyzed by western-blotting method after reduction with mercaptoethanol and ELISA method.

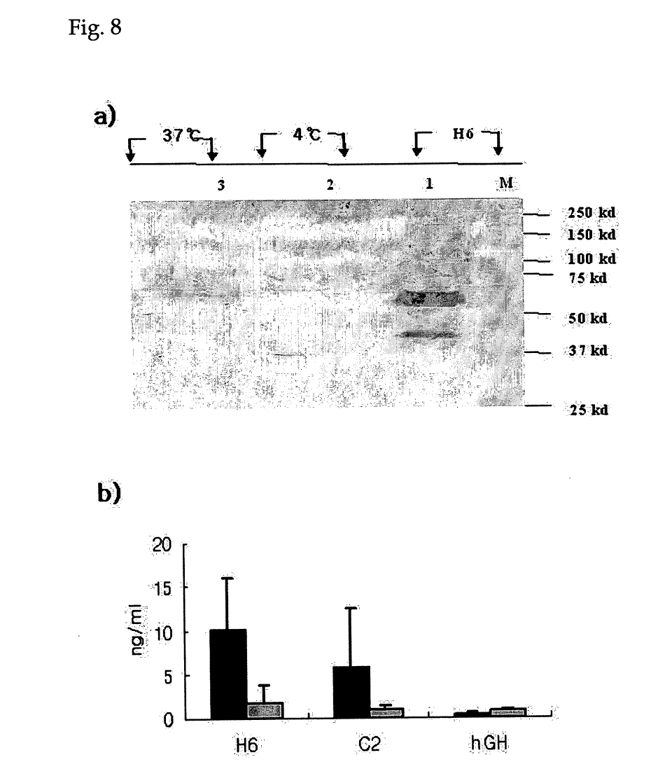

In FIG. 8a, H6, fusion protein hGH-Fc transported across T84 monolayer at 37° C. was detected by western blotting method (Lane3), but the protein transported across T84 monolayer at 4° C. was not detected (Lane2).

In FIG. 8b, H6(hGH-Fc), C2(hGH-Fc without hinge) fusion proteins showed the more efficient transcytotic functional activity after 1 hr incubation at 37° C. respectively (dark gray bar) than hGH control (column 6) but their transcytotic activities were very low after 1 hr incubation at 4° C.

Then, to test the human growth hormone activity of the chimeric proteins prepared the human hepatoma cell line, HepG2 cells (ATCC) which have the human growth hormone receptor and could increase release of IGF1. HepG2 cells were grown at 37° C. in 5% CO₂ added, in 24 well plate with surface area of 1.9 cm² for IGF1 measurement. The culture medium was Dulbecco's Modified Eagle's Medium without phenol red, supplemented with 10% heat-inactivated fetal calf serum, 2 mM L-glutamine, 1 mM non-essential amino acids and 100 U/ml streptomycin (all from Gibco). The HepG2 cells were grown to subconfluency for 3 days. The subconfluent cell layers were washed twice with phosphate-buffered saline (PBS, from Gibco), after which the cells were cultivated for 1 day under serum-free conditions. The medium was then replaced by fresh serum-free medium, and the cells were cultivated in the presence or absence of the chimeric proteins for 2 days. The chimeric proteins and human growth hormone prepared as the above were added as a single dose. Solvent only was added to the control wells. The experiments for IGF1 measurement were replicated 5 times. At the end of the incubation, the media were removed and stored at −70° C. until assayed.

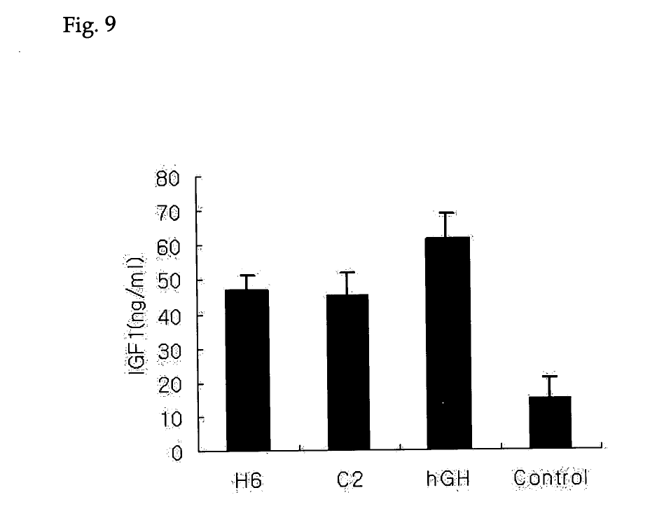

In FIG. 9, human growth hormone and chimeric proteins, IgG fused with human growth hormone, all stimulated IGF1 release from HepG2 cells at concentration of 1 pM/ml. These concentrations were 2-3 fold higher than IGF1 concentration of control wells. The cell viability at the end of the experiment did not differ from control wells.

Accordingly, the inventors named Pichia pastoris C2 for yeast transformant producing chimeric human growth hormone fused with CH2-CH3 domain of Fc fragment of human IgG which have transcytotic activity and promote release of IGF1 by production of human growth hormone, and deposited the strain to Korean Agricultural Culture Collection (address: 225, Seodun-dong, Kweonseon-ku, Suwon 441-707, Republic of Korea; phone: 82-31-299-1794; facsimile: 82-31-299-1798) on Sep. 20, 2004 with accession number KACC 93017.

Also, the inventors named Pichia pastoris H6 for yeast transformant producing chimeric human growth hormone fused with hinge-CH2-CH3 domain of Fc fragment of human IgG which have transcytotic activity and promote secretion of IGF1, and deposited the strain to Korean Agricultural Culture Collection (address: 225, Seodun-dong, Kweonseon-ku, Suwon 441-707, Republic of Korea; phone: 82-31-299-1794; facsimile: 82-31-299-1798) on Sep. 20, 2004 with accession number KACC 93018.

Mode for Carrying Out the Invention

### EXAMPLE II

### Activity Transcytosis and Human Growth Hormone of Chimeric Protein Expressed on Lactobacillus

As disclosed in the above, fusion protein H6 showed similar transcytotic activity with that of growth hormone promoting production of IGF1 in human hepatoma cell. Accordingly, to test the transcytotic capacity of the protein produced by Lactobacillus brevis prepared Lactobacillus expressing H6 fusion protein, and tested the transcytotic capacity of the chimeric protein produced from transformants in T84 using transwell system.

For this, T84 monolayers were incubated on 3.0 μm pore sized transwell (from corning costar) until they were exhibited high electrical resistances(800-1,200Ω/cm¹) in DMEM/F12 supplemented with 5% fetal calf serum (all from Gibco). The T84 cells were grown to subconfluency and were co-cultivated with Lb. H6 at 1×10⁶ cell/ml and with Lactobacillus brevis as control for 1 day. An aliquot of the contralateral well medium was collected and were analyzed by ELISA method and were splitted on MRS agar plate supplemented with 5 ng/ml chloramphenicol to test the invasion by Lactobaillus strains.

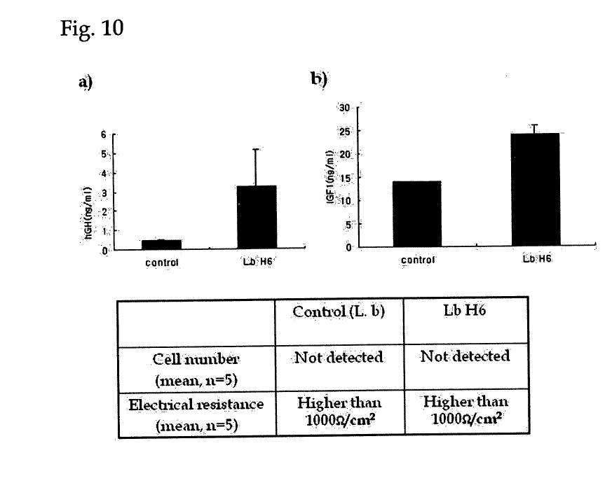

FIG. 10a showed that the protein, hGH-Fc of IgG produced by Lb. H6 could be transported throughout the human intestinal monolayer efficiently and there was no invasion of Lactobacillus strains across T84 monolayer and T84 monolayer used in this experiment were exhibited high electrical resistances (800-1,200Ω/cm²) still.

Then, the IGF1 production response of fusion protein produced from Lb. H6 by co-cultivating on HepG2 cells with Lb. H6 strain or Lactobacillus brevis for 1 day respectively was tested. HepG2 cells were grown at 37° C. in 5% CO₂ added, in 24 well plate with surface area of 1.9 cm² for IGF1 measurement. The culture medium was Dulbecco's Modified Eagle's Medium without phenol red, supplemented with 10% heat-inactivated fetal calf serum, 2 mM L-glutamine, 1 mM non-essential amino acids and 100 U/ml of streptomycin (all from Gibco). At the end of the incubation, the media were removed and stored at −70° C. until assayed.

As shown in FIG. 10b, fusion protein, IgG fused with human growth hormone produced by Lb. H6 strain stimulated IGF1 release from HepG2 cells. Its IGF1 concentration was 2 fold higher than IGF1 concentration of control wells which were co-cultivated with Lactobacillus brevis. The cell viability at the end of the experiment did not differ from control wells.

### EXPERIMENTAL EXAMPLE I

### In Vivo Bioassay of the Probiotic Lactobacillus Producing Chimeric Human Growth Hormone for Oral Use in Rats

We confirmed the function of the fusion protein produced by Lb. H6 by in vitro experiment of Example I and II. To test the function of the fusion protein produced by Lb. H6 in vivo, we administered the Lb. H6(L. brevis transformant) producing the fusion protein orally trice to total twenties rats of 4 week old-male Strague Dawley rat (average weight of rat: 32 g) composed of five rats per experimental group, and measured the human growth hormone-hinge-Fc fusion protein (H6). The above experimental groups composed of a group administered with media only (M), a group administered with Lactobacillus brevis (Lb) and a group administered with Lb. H6 producing H6 fusion protein (Lb. H6). The feeds were given freely into rats, adaptation period for 1 week after administration was given, and then change in weight gain of rats was tested for 3 weeks. The feeds were given twice per 1 day, that is, at am 10:00 and pm 5:00 and light/dark cycle was set by 12 hrs respectively. Medium, Lactobacillus and transformant Lactobacillus were administrated by 5 ml per a rat intragastrically using a ball-point needle altogether with weighting before feeding into the rats in the morning of starting date of the test. The rats were weighted every morning before feeding for 21 days. To assay hormone concentration in blood, blood collected from heart was centrifuged at 3000 xg for 20 min and the resulted blood plasma was stored in −80° C. until assayed. hGH and IGF1 concentration in blood was assayed by RIA (Radio Immunoassay) method.

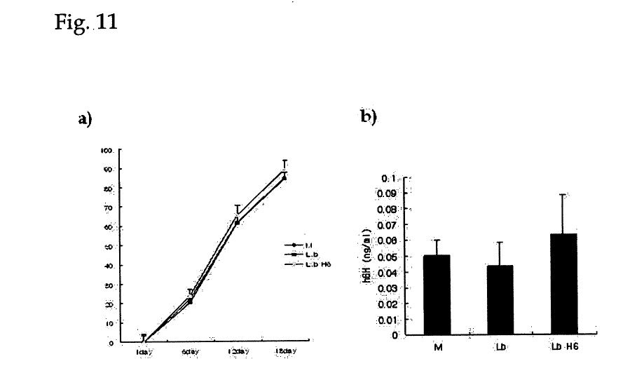

As shown in FIG. 11, the human growth hormone concentration in blood of rats to which are administered the Lb. H6 is not significantly different from that of control. However, the weight gain of rats to which are administered the Lb. H6 is higher than that of control although there is no significant difference.

TABLE 1
Weight gain of the rats to which are administered the Lb. H6

| Weight (g) | Control | Lb | Lb. H6 |
|---|---|---|---|
| Initial | 64.08 | 64.357 | 65.41 |
| Finial | 161.18 | 164.31 | 165.2 |

As a result, probiotic Lactobacillus and yeast producing human growth hormone fused with Fc fragment of human IgG according to the present invention showed transcytotic activity on incubation at 37° C. and promoted production of IGF1. That is, these microorganisms can be used treatment of patients with growth hormone deficiency syndrome, because they deliver and express chimeric protein safely into intestine by oral route.

From the above result, the inventors named Lactobacillus brevis H6 for Lactobacillus transformant producing human growth hormone fused with hinge-CH2-CH3-domain of Fc fragment of human IgG which has transcytotic activity and promotes release of IGF1 and increases weight gain of rats, and deposited the strain to Korean Agricultural Culture Collection(address: 225, Seodun-dong, Kweonseon-ku, Suwon 441-707, Republic of Korea; phone: 82-31-299-1794; facsimile: 82-31-299-1798) on Sep. 20, 2004 with accession number KACC 91137.

What is claimed is:

1. A recombinant Lactobacillus expression vector which named pNZ123(1-5 N) and illustrated by the map of FIG. 2, comprising human growth hormone coding gene conjugated with hinge-CH2-CH3 domain of Fc fragment of human IgG coding gene, in which the vector is derived from plasmid pNZ123 obtained from Lactobacillus brevis ATCC 8287.

2. A Lactobacillus brevis H6(accession number: KACC 91137) expressing a chimeric protein which is human growth hormone fused with hinge-CH2-CH3 domain of Fc fragment of human IgG.

3. A Pichia pastoris H6(accession number: KACC 93018) expressing the chimeric protein which is human growth hormone fused with hinge-CH2-CH3 domain of Fc fragment of human IgG.

4. A Pichia pastoris H6(accession number: KACC 93017) expressing a chimeric protein, which is human growth hormone fused with CH2-CH3 domain of Fc fragment of human IgG.

5. A chimeric protein having transcytotic activity, in which is expressed from a recombinant gene comprising human growth hormone conjugated with hinge-CH2-CH3 domain of Fc fragment of human IgG.
